(19) United States
(12) Patent Application Publication
(10) Pub. No.: US 2024/0233861 A1
(43) Pub. Date: Jul. 11, 2024

(54) Systems and Methods to Determine RNA Structure and Uses Thereof

(71) Applicant: The Board of Trustees of the Leland Stanford Junior University, Stanford, CA (US)

(72) Inventors: Raphael Townshend, Stanford, CA (US); Stephan Eismann, Stanford, CA (US); Andrew Watkins, Stanford, CA (US); Rhiju Das, Palo Alto, CA (US); Ron O. Dror, Stanford, CA (US)

(73) Assignee: The Board of Trustees of the Leland Stanford Junior University, Stanford, CA (US)

(21) Appl. No.: 18/562,693

(22) PCT Filed: May 20, 2022
(86) PCT No.: PCT/US2022/072483

§ 371 Date: Nov. 20, 2023

Related U.S. Application Data
(60) Provisional application No. 63/196,637, filed on Jun. 3, 2021.
(60) Provisional application No. 63/191,175, filed on May 20, 2021.

(51) Int. Cl. G16B 15/30; G16B 15/10; G16B 40/20

(52) U.S. Cl. CPC G16B 15/30; G16B 15/10; G16B 40/20

(57) ABSTRACT

Embodiments herein describe systems and methods to determine RNA structure and uses thereof. Many embodiments utilize one or more machine learning models to determine an RNA structure. In various embodiments, the machine learning model is trained using experimentally determined RNA structures. Certain embodiments identify one or more ligands or drugs that bind to an RNA structure, which can be used to treat an individual for a disease, disorder, or infection. Various embodiments determine structure of other molecules, including DNA, proteins, small molecules, etc. Further embodiments determine interactions between multiple molecules and/or molecule types (e.g., RNA-RNA interactions, RNA-DNA interactions, DNA-protein interactions, etc.)

### CROSS-REFERENCE TO RELATED APPLICATIONS

The current application claims priority to U.S. Provisional Patent Application No. 63/191,175 entitled “Geometric Deep Learning of RNA Structure” to Townshend et al., filed May 20, 2021 and U.S. Provisional Patent Application No. 63/196,637 entitled “Systems and Methods to Determine RNA Structure and Uses Thereof” to Townshend et al., filed Jun. 3, 2021; the disclosures of which are hereby incorporated by reference in their entireties.

### STATEMENT REGARDING FEDERALLY SPONSORED RESEARCH OR DEVELOPMENT

This invention was made with Government support under contract W911NF-16-1-0372 awarded by the Department of the Army; under contract DE-AC02-76SF00515 awarded by the Department of Energy; and under contracts CA219847 and GM122579 awarded by the National Institutes of Health. The Government has certain rights in the invention.

### FIELD OF THE INVENTION

The present invention relates to determining RNA structure; more specifically, the present invention relates to systems and methods incorporating machine learning to determine RNA structure based on RNA sequence.

### BACKGROUND

RNA molecules—like proteins—fold into well-defined three-dimensional (3D) structures to perform a wide range of cellular functions, such as catalyzing reactions, regulating gene expression, modulating innate immunity, and sensing small molecules. Knowledge of these structures is extremely important for understanding the mechanisms of RNA function, designing synthetic RNAs, and discovering RNA-targeted drugs. General knowledge of RNA structure lags far behind that of protein structure: the fraction of the human genome transcribed to RNA is approximately 30-fold larger than that coding for proteins, but less than 1% as many structures are available for RNAs as for proteins. (See e.g., H. M. Berman et al., The Protein Data Bank, (available at rosb.org); the disclosure of which is hereby incorporated by reference in its entirety.) Computational prediction of RNA 3D structure is thus of tremendous interest.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In some aspects, the techniques described herein relate to a method for determining RNA structure, including obtaining an experimentally determined RNA structure, training a machine learning model with the experimentally determined RNA structure, providing an RNA sequence to the trained machine learning model, and determining an RNA structure for the RNA sequence with the trained machine learning model.

In some aspects, the techniques described herein relate to a method, where the machine learning model is a geometric deep learning neural network.

In some aspects, the techniques described herein relate to a method, where the machine learning model is an equivariant neural network including an equivariant layer.

In some aspects, the techniques described herein relate to a method, where the equivariant layer passes on rotational information to the next layer in the machine learning model.

In some aspects, the techniques described herein relate to a method, where the equivariant layer passes on translational information to the next layer in the machine learning model.

In some aspects, the techniques described herein relate to a method, where the equivariant layer includes at least one of a radial function and an angular function.

In some aspects, the techniques described herein relate to a method, where the radial function encodes distances between atoms.

In some aspects, the techniques described herein relate to a method, where the angular function considers orientations between atoms.

In some aspects, the techniques described herein relate to a method, where the equivariant neural network further includes at least one of a self-interaction layer, a pointwise normalization layer, a pointwise normalization layer, and a fully connected layer.

In some aspects, the techniques described herein relate to a method, where training the machine learning model includes sampling a training set of RNA molecules.

In some aspects, the techniques described herein relate to a method, where the training set of RNA molecules includes three-dimensional coordinates and chemical element type of each atom in each RNA molecule in the training set of RNA molecules.

In some aspects, the techniques described herein relate to a method, where sampling is selected from FARFAR2 and Monte Carlo sampling.

In some aspects, the techniques described herein relate to a method, where training the machine learning model includes optimizing the machine learning model.

In some aspects, the techniques described herein relate to a method, where optimizing the machine learning model includes selecting model parameters based on a lowest root mean square deviation (RMSD) between a predicted structure and its experimentally determined structure.

In some aspects, the techniques described herein relate to a method, where the training set includes RNA molecules of 17-47 nucleotides.

In some aspects, the techniques described herein relate to a method, where training the machine learning model further includes benchmarking the machine learning model with a benchmarking set of RNA molecules.

In some aspects, the techniques described herein relate to a method, where the benchmarking set includes RNA molecules of 27-188 nucleotides.

In some aspects, the techniques described herein relate to a method, further including obtaining a structure for a ligand and docking the ligand to the determined RNA structure to identify if the ligand binds to the RNA sequence.

In some aspects, the techniques described herein relate to a method, further including providing the ligand to an individual.

In some aspects, the techniques described herein relate to a method, where the determined RNA structure includes both secondary and tertiary structures.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A illustrates details of machine learning models in accordance with various embodiments.

FIG. 1B illustrates an exemplary training set of RNA molecules in accordance with various embodiments.

FIG. 1C illustrates a process to perform structure prediction, where various embodiments score candidate structural models, selecting the models which an embodiment predicts to be most accurate (i.e., lowest RMSD) in accordance with various embodiments.

FIGS. 1D-1E illustrate exemplary benchmarking sets of RNA molecules, most of which are much larger than any of those used for training, in accordance with various embodiments.

FIGS. 2A-2D illustrate exemplary data showing performance of machine learning models in accordance with various embodiments.

FIGS. 3A-3C illustrate exemplary data showing how embodiments can produce state-of-the-art results in blind RNA structure prediction in accordance with various embodiments.

FIGS. 4A-4B illustrates how certain embodiments learn to identify key characteristics of RNA structure that are not specified in advance in accordance with various embodiments.

FIG. 5 illustrates a method for virtual screening in accordance with various embodiments.

FIG. 6 illustrates a block diagram of components of a processing system in a computing device that can be used to predict an RNA structure in accordance with various embodiments.

FIG. 7 illustrates a network diagram of a distributed system to predict an RNA structure in accordance with various embodiments.

FIG. 8A illustrates an exemplary schematic of a neural network in accordance with various embodiments.

FIGS. 8B-8C illustrate exemplary radial (FIG. 8B) and angular (FIG. 8C) functions that are modeled in accordance with various embodiments.

### DETAILED DESCRIPTION

Despite decades of intense effort, predicting the 3D structure of RNAs remains a grand challenge, having proven more difficult than prediction of protein structure. For proteins, state-of-the-art prediction methods leverage sequences or structures of related proteins. (See e.g., D. S. Marks et al., PLOS One. 6, e28766 (2011); A. W. Senior et al., Nature. 577, 706-710 (2020); and H. Kamisetty, S. Ovchinnikov, D. Baker, Proc. Natl. Acad. Sci. U.S.A 110, 15674-15679 (2013); the disclosures of which are hereby incorporated by reference in their entireties.) Such methods succeed much less frequently for RNA, both because template structures of closely related RNAs are available far less frequently and because sequence coevolution information provides less information about tertiary contacts in RNAs. Moreover, designing a scoring function that reliably distinguishes accurate structural models of RNA from less accurate ones has proven difficult, because the characteristics of energetically favorable RNA structures are not sufficiently well understood.

This problem raises the question of whether an algorithm could learn from known RNA structures to assess the accuracy of structural models of unrelated RNAs. Such a machine learning task poses two major challenges: (1) avoiding assumptions about which structural characteristics might distinguish accurate models from less accurate ones, and (2) learning from the limited number of RNA structures that have been determined experimentally. Deep learning methods that do not require pre-defined features have led to dramatic recent advances in many fields, but their success has largely been restricted to domains where data is plentiful. (See e.g., Y. LeCun, Y. Bengio, G. Hinton, Nature. 521, 436-444 (2015); the disclosure of which is hereby incorporated by reference in its entirety.)

Many embodiments described herein tackle a particularly challenging geometric learning problem, in that they (1) learn entirely from atomic structure, using no other information (e.g., sequences of related RNAs or proteins), and (2) make no assumptions about what structural features might be important, taking inputs specified simply as atomic coordinates and without even being provided basic information such as the fact that RNAs comprise chains of nucleotides.

To accomplish this task, many embodiments are able to encode detailed geometric patterns while also automatically being able to recognize and compose them at different positions and orientations. This ability is achieved through a property known as equivariance. A function ƒ applied to a vector [symbol] is rotationally (or translationally) equivariant if rotating (or translating) its input vector is equivalent to multiplying its output by a square matrix D, which is a function of the applied transformation R:

$$f ( R \cdot \vec{x} ) = D ( R ) \cdot f ( \vec{x} )$$

It should be noted that invariance is a special case of equivariance, where the output remains unchanged upon transformation (i.e., D(R)=I). (See e.g., T. S. Cohen, M. Welling, Proceedings of International Conference on Machine Learning (2016), pp. 2990-2999; the disclosure of which is hereby incorporated by reference in its entirety.)

Additionally, certain embodiments are capable of identifying ensemble conformations, such as conformations that vary with temperature, pH, ionic conditions, etc. Some embodiments predict local and/or global quantities such as, without limitation, flexibility and energetic favorability.

Additional embodiments are also used in further methods, where identifying molecular structure is important or useful, including (but not limited to) virtual screening, lead optimization, and target identification.

### Machine Learning Models

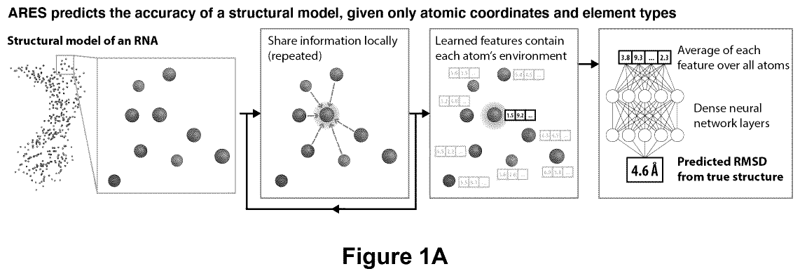

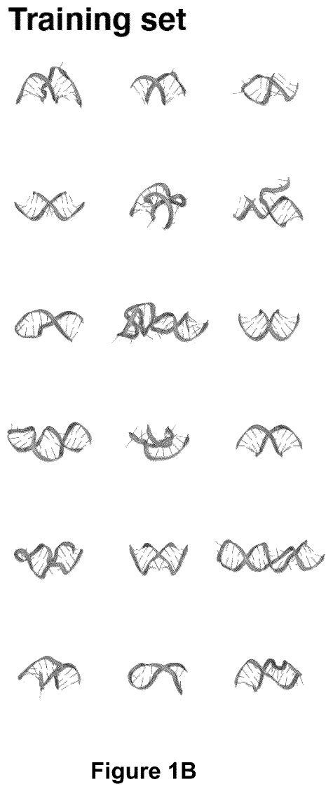

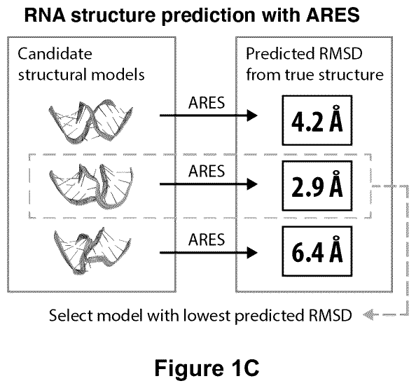

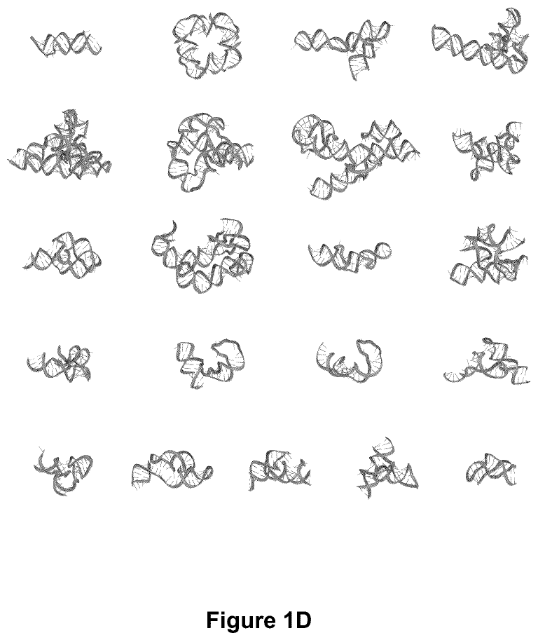

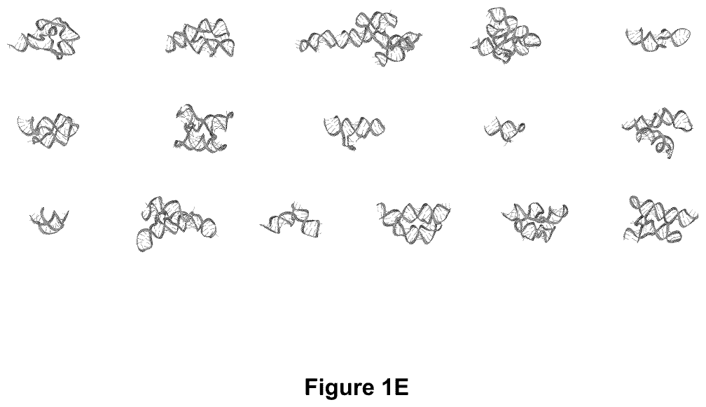

Turning to FIG. 1A, many embodiments are directed to machine learning models to address the challenges previously noted. Various embodiments implement a neural network to address the above challenges. Given a structural model (e.g., specified by the 3D coordinates and chemical element type of each atom), numerous embodiments predict the model's root mean square deviation (RMSD) from the unknown true structure. Specifically, FIG. 1A illustrates how many embodiments take a structural model as input, specified by each atom's element type and 3D coordinates. In numerous embodiments, atom features are repeatedly updated based on features of nearby atoms. As illustrated in FIG. 1A, this process results in a set of features encoding each atom's environment. Each of these features can then be averaged across all atoms, and the resulting averages can be fed into additional neural network layers, which output the predicted RMSD of the structural model from the true structure of the RNA molecule.

In certain embodiments, the machine learning model is a deep neural network comprising multiple processing layers, which each layer's outputs serving as the next layer's inputs. In such embodiments, the architecture enables the model to learn directly from 3D structures and to learn effectively given a very small amount of experimental data. Certain embodiments use other machine learning algorithms such as, without limitation, SVMs, random forests, decision trees, linear and logistic regressions, and other deep neural networks. Certain embodiments augment the neural network such as, without limitation, the use of attention-based mechanisms (e.g., transformers), residual layers, hierarchical coarse-graining, regularization, and other activation and normalization layers.

Certain embodiments use multiple different secondary structure predictions such as, without limitation, in the generation of candidate structural models, which can be used to make different final predictions. Additionally, some embodiments use multiple different templates such as in the generation of candidate structural models. Additional embodiments use coarser-grained and finer-grained models of molecular structure as input and/or output.

Various embodiments do not incorporate any assumptions about what features of a structural model are relevant to assessing its accuracy. For example, many embodiments have no preconceived notion of double helices, base pairs, nucleotides, or hydrogen bonds. It should be noted that embodiments are not restricted to RNA, and several embodiments are applicable to any type of molecular system, including (but not limited to) RNA, DNA, proteins, carbohydrates, and other molecule types.

In many embodiments, the initial layers of networks of various embodiments are designed to recognize structural motifs, whose identities are learned during the training process rather than specified in advance. In such embodiments, each of these layers computes several features for each atom based on the geometric arrangement of surrounding atoms and the features computed by the previous layer (e.g., each atom's environment). In certain embodiments, the first layer's only inputs are the three-dimensional coordinates and chemical element type of each atom. Such a strategy allows various embodiments to predict a global property (e.g., accuracy of the structural model) while capturing local structural motifs and interatomic interactions in detail.

In numerous embodiments, the architecture of these initial network layers recognizes that instances of a given structural motif are typically oriented and positioned differently from one another, and that coarser-scale motifs (e.g., helices) often comprise particular arrangements of finer-scale motifs (e.g., base pairs). In many embodiments, each layer is rotationally and translationally equivariant—that is, rotation or translation of its input leads to a corresponding transformation of its output. Equivariance captures the invariance of physics to rotation or translation of the frame of reference but ensures that orientation and position of an identified motif (or structure) are passed on to the network's next layer, which can use this information to recognize coarser-scale motifs. Equivariance allows a single filter to learn to recognize a pattern in any orientation (as the rotated pattern corresponds to multiplying the output of the filter by a square matrix), and then for those patterns to be themselves combined together in rotation-independent ways, while still being able to reason about the rotation of the subunits.

The design of these initial layers builds on recently developed machine learning techniques that capture rotational as well as translational symmetries, particularly Tensor Field Networks. (See e.g., D. E. Worrall, S. J. Garbin, D. Turmukhambetov, G. J. Brostow, Proceedings of IEEE Conference on Computer Vision and Pattern Recognition (2017), pp. 7168-7177; B. Anderson, T. Hy, R. Kondor, Advances in Neural Information Processing Systems (2019), pp. 14537-14546; M. Weiler, M. Geiger, M. Welling, W. Boomsma, T. Cohen, Advances in Neural Information Processing Systems (2018), pp. 10381-10392; N. Thomas et al., arXiv 1802.08219 [cs.LG] (2018); and S. Eismann et al., Proteins. 89, 493-501 (2020); the disclosures of which are hereby incorporated by reference in their entireties.) In many embodiments, one of the primary equivariant layers is the equivariant convolution.

### Model Training

To train various embodiments, a library of RNA structures is obtained. FIG. 1B illustrates one exemplary embodiment, RNA molecules whose experimentally determined structures were published between 1994 and 2006 were used as the training set. (See e.g., R. Das, D. Baker, Proc. Natl. Acad. Sci. U.S.A 104, 14664-14669 (2007); the disclosure of which is hereby incorporated by reference in its entirety.) For this embodiment, the RNAs in the training set comprise 17-47 nucleotides (median 26 nucleotides). Certain embodiments generate structural (e.g., 3D position of each element in the structure) models of each RNA (e.g., 100 structural models, 250 structural models, 500 structural models, 1,000 structural models, or more). Various embodiments utilize a sampling method, such as the Rosetta FARFAR2 sampling method, without making any use of the known structure. (See e.g., A. M. Watkins, R. Rangan, R. Das, Structure. 28, 963-976.e6 (2020); the disclosure of which is hereby incorporated by reference in its entirety.) Additional embodiments utilize other sampling methods, such as Monte Carlo sampling. Further embodiments then optimize the parameters of the model (e.g., neural network) such that its output matches as closely as possible the RMSD of each predicted structure from the corresponding experimentally derived structure. FIG. 1C illustrates an optimization process of an exemplary embodiment, “ARES,” where model parameters are selected based on lowest RMSD between a candidate (or predicted) structure and its true (or experimentally determined) structure.

Many embodiments assess the ability of models to identify accurate structural models of previously unseen RNAs. In doing so, various embodiments utilize a benchmark set comprising a set of RNA sequences for which experimentally determined structures have been published, but are not used in the training set. (See e.g., Z. Miao et al., RNA. 26, 982-995 (2020); the disclosure of which is hereby incorporated by reference in its entirety.) FIGS. 1D-1E illustrate benchmark sets of RNA structures used in exemplary embodiments. In FIGS. 1D-1E, each of the structures in the benchmark sets is generally larger than the structures utilized in the training set (e.g., FIG. 1B). For this exemplary embodiment, the RNAs in the benchmark sets comprise 27-188 nucleotides (median 75, with 31 of 37 RNAs comprising more nucleotides than any RNA in the training set). Various embodiments utilize a set of structural models for each RNA in the benchmark set (e.g., 100 structural models, 250 structural models, 500 structural models, 1000 structural models, 1,500 structural models, or more). In some embodiments, the benchmark set comprises RNA sequences that are longer (e.g., more nucleobases) and/or comprise larger structures than in the training set. Certain embodiments use a trained model to generate a score for each model (e.g., a predicted RMSD of each model from the native structure).

### Model Performance

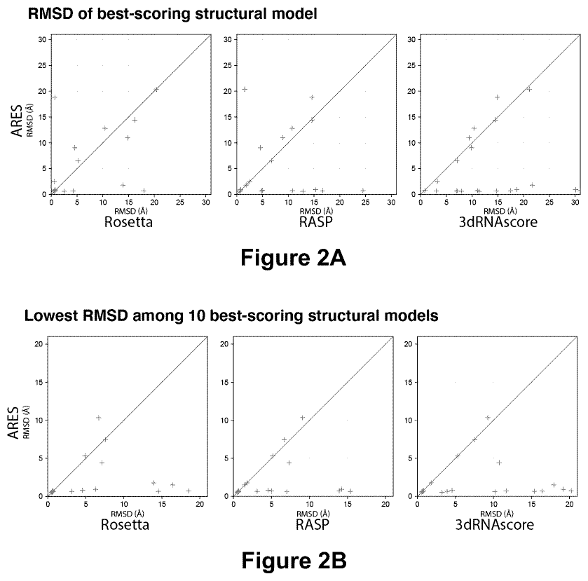

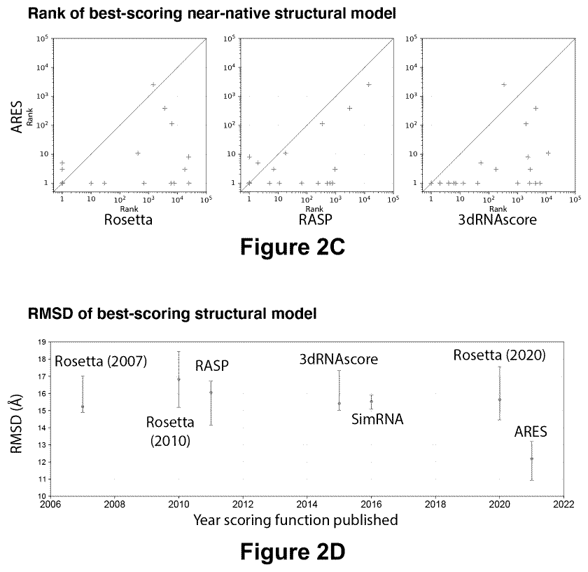

Turning to FIGS. 2A-2C, scores generated by neural networks of various embodiments can further be compared to other RNA structure prediction functions, such as Rosetta, RASP, and 3dRNAscore. (See e.g., A. M. Watkins, R. Rangan, R. Das, Structure. 28, 963-976.e6 (2020); E. Capriotti, T. Norambuena, M. A. Marti-Renom, F. Melo, Bioinformatics. 27, 1086-1093 (2011); and J. Wang, Y. Zhao, C. Zhu, Y. Xiao, Nucleic Acids Res. 43, e63 (2015); the disclosures of which are hereby incorporated by reference in their entireties.) Specifically, FIGS. 2A-2C illustrate exemplary data of one embodiment, “ARES,” as compared to Rosetta, RASP, and 3dRNAscore. Specifically, FIG. 2A illustrates a comparison of candidate structures by RMSD from ARES and each of the other structure prediction functions. In FIG. 2A, the structural model scored as best by ARES is usually more accurate (as assessed by RMSD from the native structure) than the model scored as best by the other scoring functions. The single best-scoring structural model is near-native (<2 Å RMSD) for 62% of the benchmark RNAs when using ARES, compared to 43%, 33%, and 5% for Rosetta, RASP, and 3dRNAscore, respectively. Similarly, FIG. 2B illustrates exemplary data of the 10-best scoring structural models by ARES as compared to the other scoring functions, indicating the exemplary embodiment provides an accurate structural model more frequently than when using the other scoring functions. The 10 best-scoring models include at least one near-native model for 81% of the benchmark RNAs when using ARES, compared to 48%, 48% and 33% for Rosetta, RASP and 3dRNAscore, respectively. FIG. 2C provides exemplary data of a rank of the best scoring structural model-how far down a ranked list of structures to find a near native (RMSD<2 Å)—as provided by ARES versus other scoring functions. As illustrated in FIG. 2D, the rank is usually lower (better) for ARES than for the other scoring functions. Across the RNAs, the mean rank of the best-scoring near-native model is 3.6 for ARES, compared to 73.0, 26.4 and 127.7 for Rosetta, RASP and 3dRNAscore, respectively.

Additionally, many current methods for sampling candidate structural models often fail to generate near-native models in a reasonable amount of compute time. When compared to a second benchmark that includes no near-native models, embodiments continue to outperform current methods. When predicting RNA structure, experts can often find some known structures that can be used as local templates, or other published experimental data that provides information on local tertiary structure. When benchmarked against structurally diverse RNAs, all substantially different from any of those used to train ARES or those in a previous benchmark set, and each including one or more of the following hallmarks of structural complexity: ligand binding sites, multiway junctions, and tertiary contacts. FIG. 2D illustrates exemplary data showing the exemplary embodiment “ARES” against six other scoring functions that have seen widespread use over the past 14 years. Specifically, ARES again outperforms all the other scoring functions on this second benchmark. The median RMSD across RNAs of the best-scoring structural model is significantly lower for ARES than for any other scoring function. The same is true when considering the most accurate of the 10 best-scoring structural models for each RNA.

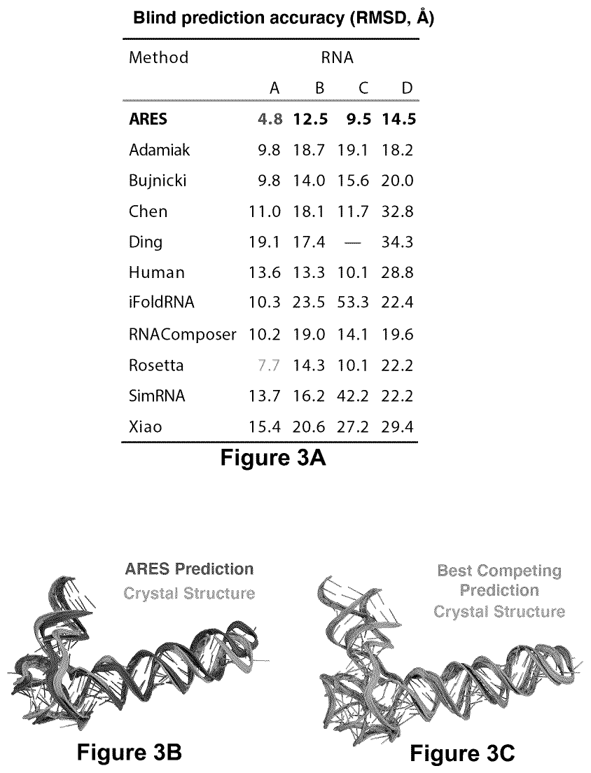

Turning to FIGS. 3A-3C, exemplary data showing how embodiments achieve state-of-the-art results in blind RNA structure prediction is illustrated—in particular, how an exemplary embodiment yielded the most accurate model as measured both by RMSD and by deformation index. Specifically, FIG. 3A illustrates structural models that the exemplary embodiment, “ARES,” selected from sets of candidates generated by to four recent rounds of the RNA-Puzzles blind structure prediction challenge: RNA A (the Adenovirus VA-I RNA), RNA B (the Geobacillus kaustophilus T-box discriminator-tRNAGly), RNA C (the Bacillus subtilis T-box-tRNAGly), and RNA D (the Nocardia farcinic T-box-tRNAlle). In the exemplary embodiment for which data is illustrated, the RNAs comprise 112-230 nucleotides (median 152.5 nucleotides). In all four (PDB codes, A: 6OL3, B: 6PMO, C: 6POM, D: 6UFM), The ARES embodiment produced the most accurate structural model of the methods tested. Competing submissions were produced by at least nine other methods for each round, including methods that used the same sets of candidate-sampled structural models but selected among them using the judgment of human experts or the Rosetta scoring function. The ARES scoring function outperforms a variety of other scoring functions applied to the same sets of candidate models, including a recent machine learning approach based on a convolutional neural network. (See e.g., J. Li et al., PLOS Comput. Biol. 14, e1006514 (2018); the disclosure of which is hereby incorporated by reference in its entirety.)

In FIGS. 3B-3C illustrate an overlay between a structural prediction of the Adenovirus VA-I RNA as compared to its experimentally determined structure, where FIG. 3B illustrates the overlay from the ARES embodiment having a 4.8 Å RMSD to the experimentally determined structure, while FIG. 3C illustrates most accurate structural model produced by any another method (Rosetta) for the Adenovirus VA-I RNA, which had an RMSD of 7.7 Å.

Additionally, certain embodiments are capable of identifying ensemble conformations, such as conformations that vary with temperature, pH, ionic conditions, etc. Further embodiments can determine structure in vivo and in vitro, where such conditions affect RNA structure.

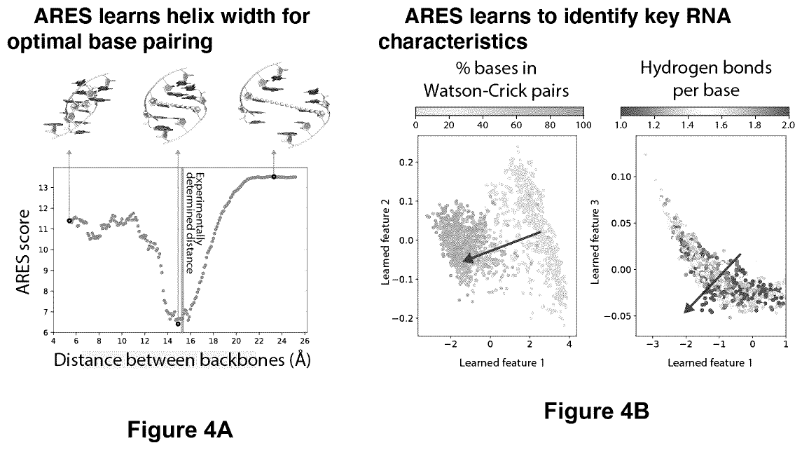

Turning to FIGS. 4A-4B, many embodiments are capable of discovering certain fundamental characteristics of RNA structure. For example, FIG. 4A illustrates exemplary data of the exemplary embodiment “ARES” correctly predicts the optimal distance between the two strands in a double helix—i.e., the distance that allows for ideal base pairing. As the distance between two complementary strands of an RNA double helix is varied, an exemplary embodiment assigns the best scores when the distance closely approximates that observed in experimental structures (vertical line in graph). Distance is measured between C4′ atoms of the central base pair (dotted lines in helix diagrams).

In addition, FIG. 4B illustrates exemplary data showing the high-level features ARES extracts from a set of RNA structures reflect the extent of hydrogen bonding and Watson-Crick base pairing in each structure, even the model was never informed that hydrogen bonding and base pairing are key drivers of RNA structure formation. Learned features separate RNA structures based on the fraction of bases forming Watson-Crick pairs (left) and on the average number of hydrogen bonds per base (right). The arrow in each plot indicates the direction of separation. Learned features 1, 2, and 3 are the 1st, 2nd, and 3rd principal components, respectively, of the activation values of the 256 nodes in ARES's penultimate layer across 1576 RNA structures.

Additionally, various embodiments also accurately identify complex tertiary structure elements, including ones that are not represented in the training data set.

The performance of many embodiments is particularly striking given that all the RNAs used for blind structure prediction (FIGS. 3A-3C) and most of those used for systematic benchmarking (FIGS. 2A-2D) are larger and more complex than those used to train exemplary embodiments (FIGS. 1A-1D).

The ability of some embodiments to outperform the previous state of the art despite using only a small number of structures for training suggests that similar neural networks could lead to substantial advances in other areas involving three-dimensional molecular structure, where data is often limited and expensive to collect. In addition to structure prediction, examples might include molecular design (both for macromolecules such as proteins or nucleic acids and for small-molecule drugs), estimating electromagnetic properties of nanoparticle semiconductors, and predicting mechanical properties of alloys and other materials.

As noted above, embodiments are capable of determining structure based only on three-dimensional molecular structure. As such, some embodiments are applicable across many other types of molecules, including (but not limited to) proteins, DNA, small molecules, polymers, antibodies, nanomaterials, and interactions between these molecules as well as interactions with RNA and any of these molecules. Certain embodiments use ligands in the prediction process such as, without limitation, including them in the generation of candidate structural models and including ligands as inputs to the neural network.

Due to the ability of embodiments to be flexible across molecule types and interactions between some molecules, further embodiments identify drugs (e.g., small molecules, biologicals, etc.) capable of binding an RNA. In certain embodiments, the drugs, which can be ligands, can be docked into an RNA structure (either experimentally discovered or determined in other embodiments) to identify candidate drugs that bind to an RNA structure. Such embodiments allow for screening of hundreds, thousands, or hundreds of thousands of small molecules or other drugs at a time.

Once drugs are identified to bind and/or how they bind to an RNA molecule, several embodiments determine binding affinity of the drug to the RNA. Additionally, once drugs are identified to bind, various embodiments perform lead optimization on the molecules. Lead optimization can include modifications to the drugs to increase binding affinity, solubility, and/or any other desirable characteristic of the drug. Various embodiments of drugs that target or have specificity for an RNA molecule can be used as therapeutics, including as antivirals against RNA-based viruses, including SARS-CoV-2.

### Drug Discovery, Virtual Screening, and Lead Optimization

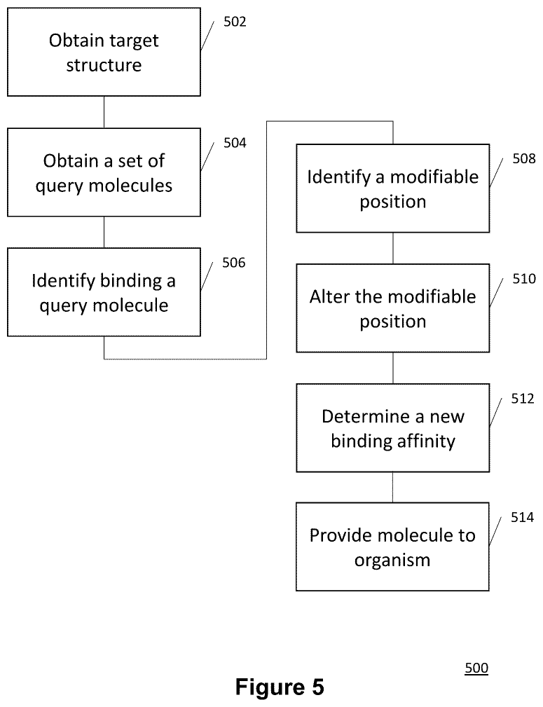

Turning to FIG. 5, various embodiments are capable of being used to find drugs, including small molecules, that bind against specific targets, such as illustrated in exemplary method 500. In such embodiments, machine learning models, such as a neural network, predict binding affinity of molecules bound to RNA structures, such as RNA aptamers, mRNA, tRNA, rRNA, DNA, and/or any other organic molecules. Various embodiments train the neural network based on experimentally derived RNA-ligand binding and structural data and/or experimentally derived RNA-ligand binding affinity data. Embodiments trained on binding and structural data can identify RNA-ligand complexes, such that the binding location can be identified or predicted, while embodiments trained on binding affinity data can identify the binding strength of RNA-ligand complexes. Certain embodiments utilize a single model or multiple models to provide both RNA-ligand complex structure and RNA-ligand binding affinity. Such embodiments are capable of virtual screening for molecules or drugs that may be effective for targeting molecules (e.g., RNA, DNA, etc.). It should be noted that while RNA-ligand complexes are described in the foregoing section, such embodiments are expansible to other molecule types, including DNA, proteins, carbohydrates, etc.

At 502, various embodiments obtain a structure of a target molecule. As noted above, such structures can include nucleic acids (e.g., RNA aptamers, mRNA, tRNA, rRNA, DNA), and/or any other organic molecules of interest. In some embodiments, such structures are obtained experimentally (e.g., from crystallography), while some embodiments obtain structures from databases, including ChEMBL, PDB, etc. Further embodiments obtain a structure from a prediction methodology, such as described herein.

At 504, many embodiments obtain a set of query molecules (e.g., drugs). The set of query molecules can include any number of molecules, including 1 molecule, 2 molecules. 3 molecules, 4 molecules, 5 molecules, 10 molecules 15 molecules, 20 molecules, 25 molecules, 50 molecules, 75 molecules, 100 molecules, or more. Many embodiments obtain structures for the query molecules including coordinates for each atom in the molecule.

At 506, many embodiments A) identify if each query molecule binds to the target molecule, B) generate a structure of the RNA-ligand complex, and/or C) generate a binding affinity for each binding molecule.

Further embodiments perform lead optimization of one or more query molecules at 508-512. In various embodiments, a modifiable location is identified on the query ligand at 508. The modifiable position can be any position that may allow for additional modification that allows for a change in chemical group, including groups that may sit internal to a binding site that could increase binding affinity, while some embodiments may identify a location that may not contribute to binding, such that a modification could be used for increasing solubility, labeling, or conjugating additional molecules to the query molecule.

At 510, some embodiments alter the modifiable position. For example, some embodiments may alter the position to increase binding affinity via the inclusion of a chemical group that may form an interaction with the target protein, such as via a hydrogen bond, salt bridge, and/or hydrophobic interaction.

Additional embodiments determine a new binding affinity for the modified query molecule at 512. Such binding affinity is assessed similarly to 506, where the pose prediction and potential demonstrate a binding affinity for the modified query molecule.

It should be noted that various embodiments may perform various steps simultaneously, multiple times, and/or omit steps as appropriate for a particular use. For example. Some embodiments may obtain multiple query ligands and/or multiple sets of known-binding ligands for use within an embodiment of method 500.

In some embodiments, when a candidate molecule is identified (e.g., at 506) or optimized (e.g., at 512), such embodiments provide 514 the molecule to an individual, or living organism, for treatment. Such treatments can include drugs that may inhibit viral infection or progression, such as for RNA-based viruses, including (but not limited to) coronaviruses (e.g., SARS-CoV-2, SARS, MERS), picornaviruses, and other viruses.

### Computer Executed Embodiments

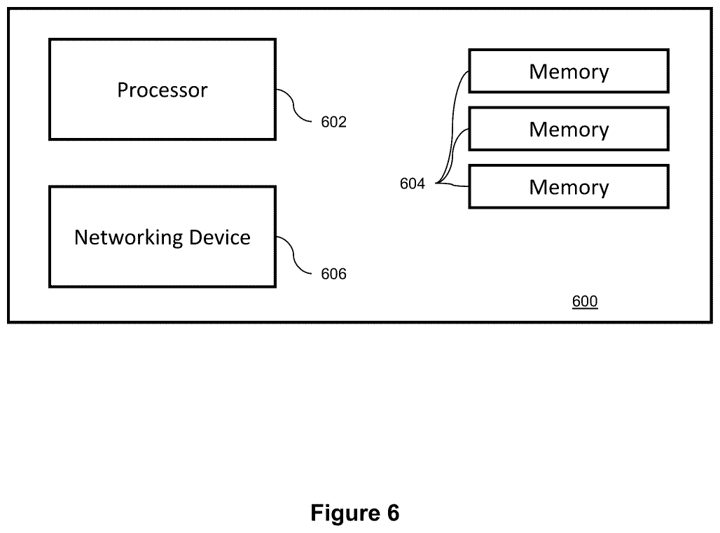

Processes that provide the methods and systems for generating a surgical risk score in accordance with some embodiments are executed by a computing device or computing system, such as a desktop computer, tablet, mobile device, laptop computer, notebook computer, server system, and/or any other device capable of performing one or more features, functions, methods, and/or steps as described herein. The relevant components in a computing device that can perform the processes in accordance with some embodiments are shown in FIG. 6. One skilled in the art will recognize that computing devices or systems may include other components that are omitted for brevity without departing from described embodiments. A computing device 600 in accordance with such embodiments comprises a processor 602 and at least one memory 604.

Memory 604 can be a non-volatile memory and/or a volatile memory, and the processor 602 is a processor, microprocessor, controller, or a combination of processors, microprocessor, and/or controllers that performs instructions stored in memory 604. Such instructions stored in the memory 604, when executed by the processor, can direct the processor, to perform one or more features, functions, methods, and/or steps as described herein. Any input information or data can be stored in the memory 604—either the same memory or another memory. In accordance with various other embodiments, the computing device 600 may have hardware and/or firmware that can include the instructions and/or perform these processes.

Certain embodiments can include a networking device 606 to allow communication (wired, wireless, etc.) to another device, such as through a network, near-field communication, Bluetooth, infrared, radio frequency, and/or any other suitable communication system. Such systems can be beneficial for receiving data, information, or input (e.g., structural data, sequence data, etc.) from another computing device and/or for transmitting data, information, or output (e.g., structural prediction) to another device.

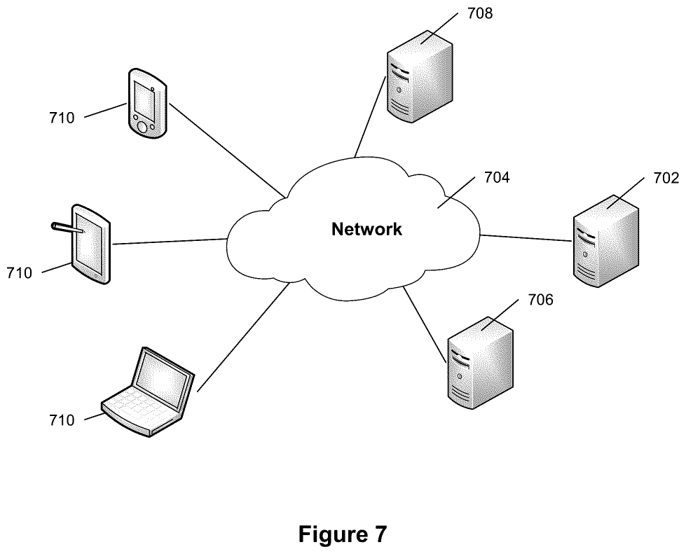

Turning to FIG. 7, an embodiment with distributed computing devices is illustrated. Such embodiments may be useful where computing power is not possible at a local level, and a central computing device (e.g., server) performs one or more features, functions, methods, and/or steps described herein. In such embodiments, a computing device 702 (e.g., server) is connected to a network 704 (wired and/or wireless), where it can receive inputs from one or more computing devices, including structural data and/or sequence data (e.g., peptide, protein, DNA, and/or RNA sequence data) from a database or repository 706, input data (e.g., one or more of RNA sequences, DNA sequences, peptide sequences, and/or protein sequences) provided from a laboratory computing device 708, and/or any other relevant information from one or more other remote devices 710. Once computing device 702 performs one or more features, functions, methods, and/or steps described herein, any outputs (e.g. predicted or computed structure) can be transmitted to one or more computing devices 706, 708, 710 for further use-including (but not limited to) manufacture or synthesis, medical treatment, and/or any other action relevant to an RNA structure. Such actions can be transmitted directly to an interested party or researcher, (e.g., via messaging, such as email, SMS, voice/vocal alert) for such action and/or entered into a database.

In accordance with still other embodiments, the instructions for the processes can be stored in any of a variety of non-transitory computer readable media appropriate to a specific application.

### EXEMPLARY EMBODIMENTS

Although the following embodiments provide details on certain embodiments of the inventions, it should be understood that these are only exemplary in nature, and are not intended to limit the scope of the invention.

### Example 1: Atomic Rotationally Equivariant Scorer (“ARES”)

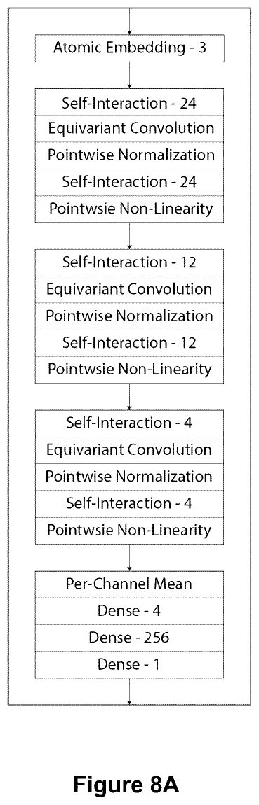

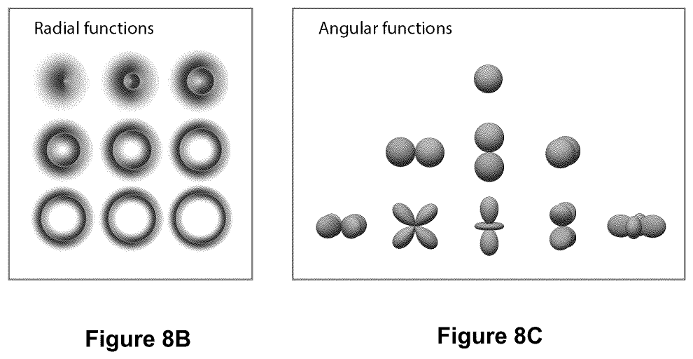

As an illustrative example, one embodiment of a machine learning model is described herein, which was used to predict RNA structure. A schematic of the model is illustrated in FIG. 8A.

### Equivariant Convolution

Equivariant convolutions take in a set of atoms in three-dimensional (3D) space, with associated feature vectors, and use both their features and relative positions and orientations to produce a new feature vector associated with each atom. This outputted vector is learnable.

For a given atom a (referred to as the source atom), the equivariant convolution is a set of functions [symbol] applied one at a time to each atom b within its local neighborhood (referred to as the neighbor atoms). Certain embodiments define [symbol] ₐₚ as the 3D vector between the source atom and a given neighbor atom. In many embodiments, functions [symbol] only take as input the vector [symbol] _{ab}, and their output is combined with a given neighbor atom's current feature vector [symbol] _{b} to produce an updated feature vector [symbol] for the source atom. In this way, a neighboring atom's information is shared with the source atom. The design of the functions [symbol] , as well how their outputs are combined with neighbor's feature vectors, is the key to ensuring the network is equivariant while still allowing for the capture of detailed geometric information.

In many embodiments, the set of functions [symbol] is composed of all possible combinations of two classes of sub-functions: radial and angular functions, such as defined herein.

### Radial Functions

The radial functions encode the distances between atoms, without considering their relative orientations. Radial functions take the form of a dense neural network, in many embodiments. The inputs [symbol] to this network are computed by applying a filter bank of Gaussians (examples illustrated in FIG. 8B) to the magnitude r_{ab}=∥ [symbol] _{ab}∥:

$$\vec{G} ( {r}_{ab} ) = [ {G}_{0} ( {r}_{ab} ) , {G}_{1} ( {r}_{ab} ) , … , {G}_{n} ( {r}_{ab} ) ]$$

$$With :$$

$${G}_{j} ( {r}_{ab} ) = \frac{1}{\sqrt{2 π σ}} {e}^{- \frac{{( {r}_{ab} - {μ}_{j} )}^{2}}{2 {σ}^{2}}}$$

Where σ=1 Å, n=11, and

$$\\ {μ}_{j} = \frac{12}{11} j Å .$$

an exemplary embodiment, the dense network has one hidden layer of dimension 12, with a ReLU activation before the hidden layer and outputs a vector of fixed size. In many embodiments, there are learnable biases for both the hidden and output layers of this dense network. The entries of the output vector provide all the radial filter outputs:

$$[ {R}_{0} ( {r}_{a b} ) , {R}_{1} ( {r}_{a b} ) , … , {R}_{C} ( {r}_{a b} ) ] = Dense ( \vec{G} ( {r}_{a b} ) )$$

Where C is the total number of radial outputs. As these functions only consider distances between atoms, they are invariant to translations and rotations.

### Angular Functions

The angular functions consider orientations between atoms, not distances. Various embodiments use real spherical harmonics Y as angular functions. Spherical harmonics are grouped by their angular resolution l∈ [symbol] , which are refered to as angular order—there are l+1 harmonics per order. To index within each order, various embodiments use an angular index m, with m∈{−l, −l+1, . . . , l−1, l}. They are applied to the unit vector \hat{r}_{ab}= [symbol] _{ab}/∥ [symbol] _{ab}∥:

$${A}_{m}^{l} ( {\hat{r}}_{a b} ) = {Y}_{m}^{l} ( {\hat{r}}_{a b} )$$

Numerous embodiments define L as the maximum order used, thus using M=Σₗ₌₀^{L}(2l+1) angular functions total. Certain embodiments use L=2, giving the zeroth-, first-, and second-order harmonics (examples illustrated in FIG. 8C). The zeroth-order harmonic can capture scalar quantities such as aromaticity or charge. The first-order harmonics can capture vector quantities, like hydrogen bond vectors or an aromatic ring's normal vector. The second-order harmonics can capture matrix quantities, like the moment of inertia for groups of atoms.

One important property of spherical harmonics is that when a rotation is applied to an input unit vector \hat{r}, a harmonic of a given order is transformed into a linear combination of harmonics of the same order. So, if the harmonics of a particular order l as a vector [symbol] ^{l}, it provides:

$$\\ \vec{Y} {}^{{}_{} l} ( R \cdot {\hat{r}}_{ab} ) = {D}^{{}_{} l} ( R ) \cdot \vec{Y} {}^{{}_{} l} ( {\hat{r}}_{ab} )$$

Where D^{l} is a matrix dependent on the rotation R known as a Wigner D-matrix. Thus, critically, spherical harmonics within a given order are equivariant to rotations.

### Combined Functions

Finally, many embodiments define [symbol] as the set of “combined functions” F resulting from every possible combination of radial and angular functions. These form the core of the equivariant convolution:

$$\\ 𝔽 ( {\vec{r}}_{ab} ) = \{ \begin{matrix}{F}_{cm}^{{}_{} l} ( {\vec{r}}_{ab} ) = {R}_{c} ( {r}_{ab} ) {A}_{m}^{l} ( {\hat{r}}_{ab} ) \vert c ∈ \{ 0 , 1 , … , C \} , \\ l ∈ \{ 0 , 1 , … , L \} , m ∈ \{ - l , - l + 1 , … , l \}\end{matrix} \}$$

C is referred to as the dimension of the equivariant convolution. The three equivariant convolutions have dimensions 24, 12, and 4. As the radial sub-function is invariant to rotations, and the angular sub-function is equivariant to rotations within an angular order, each combined function is equivariant to rotations within an angular order. Similarly, these combined functions are equivariant to translations.

Each combined function is applied to [symbol] _{ab}, and the result is multiplied with each entry i in the neighbor atom's associated feature vector [symbol] _{b} to obtain a per-function-per-neighbor output o_{bicm}^{l}:

$$\\ {o}_{bicm}^{{}_{} l} = {F}_{cm}^{{}_{} l} ( {\vec{r}}_{ab} ) \cdot {V}_{bi}$$

Where m, c, and l are the angular, radial, and order indices, and i is the feature vector index. In many embodiments, these outputs are summed over all neighboring atoms b of our source atom a to obtain a per-function output O_{aicm}^{l}.

$$\\ {O}_{aicm}^{{}_{} l} = {\sum }_{b ∈ neighbors ( a )} {o}_{bicm}^{{}_{} l}$$

these per-function activations can be combined across i, c, l, and m, to obtain a new feature vector for our source atom. This combination is not straightforward, as merging the filters spanning the different angular orders, while still maintaining equivariance, requires the use of Clebsch-Gordan coefficients.

### Clebsch-Gordan Coefficients

To understand why combining the different outputs is not straightforward, note that the activations O_{aicm}^{l} after a round of equivariant convolution are indexed by angular order. Thus, the atom's updated feature vector has different components inhabiting different angular orders. Therefore, in practice the index i is redefined into [symbol] as the corresponding angular, radial, and order indices m, c, and l:

$$\\ {V}_{acm}^{{}_{} l} ≡ {V}_{ai}$$

For the first layer, may embodiments only have features of angular order l=0, and a total of C=3 radial features, for the three possible element types encoded. For subsequent layers, trouble arises because each entry of their input vector inhabits a certain angular order, and each filter inhabits its own order as well. Thus, a per-function-per-neighbor activation now becomes:

$$\\ {o}_{{bcm}_{i} {m}_{f}}^{{l}_{i} {l}_{f}} = {F}_{{cm}_{f}}^{{l}_{f}} ( {\vec{r}}_{ab} ) \cdot {V}_{{bcm}_{i}}^{{}_{} {l}_{i}}$$

Where the ƒ and i subscript can be added to the angular order and index to denote their provenance from either the filter or the feature vector input. Note that the input vector and filters are assumed to have the same number of radial filters. In turn, a per-function activation is indexed as:

$$\\ {O}_{{acm}_{i} {m}_{f}}^{{l}_{i} {l}_{f}} = {\sum }_{b ∈ neighbors ( a )} {o}_{{bcm}_{i} {m}_{f}}^{{l}_{i} {l}_{f}}$$

Now the activations span two different orders, and so it is desirable to reduce the next layer's feature vector to a single angular order (otherwise each equivariant convolution layer would add further new dimensions), which is denoted through the subscript o. Clebsch-Gordan coefficients C are a way to combine them that is equivariant to rotations. These coefficients map two orders (input lᵢ and filter l_{ƒ}) to one (output lₒ), giving updated outputs [symbol] :

$$\\ {U}_{{acm}_{o}}^{{}_{} {l}_{o}} = {\sum }_{{m}_{i} , {m}_{f}} {C}_{( {l}_{f} , {m}_{f} ) ( {l}_{i} , {m}_{i} )}^{{}_{} ( {l}_{o} , {m}_{o} )} {O}_{{acm}_{i} {m}_{f}}^{{l}_{i} {l}_{f}}$$

Some examples of Clebsch-Gordan coefficients include:
For lᵢ=0, l_{ƒ}=0, lₒ=0: C₍ₗ_{_{ƒ}}_{,m}_{_{ƒ}}₎₍ₗ_{ᵢ}_{,m}_{ᵢ}₎^{(l}^{ₒ}^{,m}^{ₒ}⁾=1. It amounts to scalar multiplication.
For lᵢ=1, l_{ƒ}=1, lₒ=0: C₍ₗ_{_{ƒ}}_{,m}_{_{ƒ}}₎₍ₗ_{ᵢ}_{,m}_{ᵢ}₎^{(l}^{ₒ}^{,m}^{ₒ}⁾ ∝δₘ_{ᵢ}_{,m}_{_{ƒ}}, where δ is the Kronecker delta tensor. It amounts to a scaled dot product.
For lᵢ=1, l_{ƒ}=0, lₒ=1: C₍ₗ_{_{ƒ}}_{,m}_{_{ƒ}}₎₍ₗ_{ᵢ}_{,m}_{ᵢ}₎^{(l}^{ₒ}^{,m}^{ₒ}⁾ ∝δₘ_{_{ƒ}}_{,m}_{ₒ}. It amounts to scalar multiplication of a vector.
For lᵢ=0, l_{ƒ}=1, lₒ=1: C₍ₗ_{_{ƒ}}_{,m}_{_{ƒ}}₎₍ₗ_{ᵢ}_{,m}_{ᵢ}₎^{(l}^{ₒ}^{,m}^{ₒ}⁾ ∝δm_{ƒ},mₒ. It amounts to scalar multiplication of a vector.
For lᵢ=1, l_{ƒ}=1, lₒ=1: C₍ₗ_{_{ƒ}}_{,m}_{_{ƒ}}₎₍ₗ_{ᵢ}_{,m}_{ᵢ}₎^{(l}^{ₒ}^{,m}^{ₒ}⁾ ∝εₘ_{ₒ}_{,m}_{ᵢ}_{,m}_{_{ƒ}}, where & is the Levi- Civita tensor. It amounts to a cross product.

In general, Clebsch-Gordan coefficients have the constraint that |lᵢ−l_{ƒ}|≤lₒ≥lᵢ+l_{ƒ}, and thus there are only certain combinations of input, filter, and output orders that are possible.

Additional layers are described next, which are more straightforwardly equivariant to rotations as they only operate on individual atoms (atomic embedding, pointwise normalization, pointwise non-linearity, and pointwise self-interaction) or only operate on rotationally invariant features (per-channel mean and subsequent layers). Composing these individually equivariant layers together yields a network that is overall equivariant.

### Pointwise Normalization

The pointwise normalization operation acts on each atom a's feature vector [symbol] . This vector can be split by angular order and each component can be divided by its L₂ norm to obtain a new feature vector [symbol] ₐ:

$$\\ {U}_{acm}^{{}_{} l} = \frac{{V}_{acm}^{{}_{} l}}{\sqrt{{\sum }_{c , m} {( {V}_{acm}^{{}_{} l} )}^{2}}}$$

Where m, c, and l are the same angular, radial, and order indices as defined in previous layers.

### Pointwise Non-Linearity

The pointwise non-linearity operation acts on each entry of each atom's feature vector [symbol] . Many embodiments use an equivariant non-linearity adapted from Tensor Field Networks:

$$\\ {U}_{acm}^{{}_{} l} = \{ \begin{matrix}η ( {V}_{acm}^{{}_{} l} ) if l = 0 \\ {V}_{acm}^{{}_{} l} \cdot η ( \sqrt{{\sum }_{m} {( {V}_{acm}^{{}_{} l} )}^{2}} + {b}^{{}_{} l} )\end{matrix}$$

otherwise
Where b^{l} is a learnable scalar bias term (one per order), m, c, and l are the same angular, radial, and order indices as defined in previous layers, and η is a shifted soft plus non-linearity, as in SchNet:

$$\\ η ( x ) = ln ( 0.5 {e}^{{}_{} x} + 0.5 )$$

### Pointwise Self-Interaction

Many embodiments use self-interaction layers as in SchNet to mix information across radial channels between equivariant convolution layers. Such layers can be applied to each atom's features V, and split this vector by the order and index of the corresponding spherical harmonics to obtain our new feature vector [symbol] ₐ:

$$\\ {U}_{adm}^{{}_{} l} = {b}_{d} + {\sum }_{c} {V}_{acm}^{{}_{} l} {W}_{cd}$$

Where W is a learnable weight matrix, [symbol] is a learnable bias vector, m, c, and l are the same angular, radial, and order indices as defined in previous layers, and d is the new radial index. Note the bias vector is only used when operating on angular order 0 (i.e., l=0). Within a given self-interaction layer, the number of output channels d is the same for each angular order of spherical harmonics; this value is referred to as the dimension of the pointwise self-interaction. The 6 self-interaction layers have dimensions 24, 24, 12, 12, 4, and 4, respectively.

### Atomic Embedding

The atomic embedding can used to generate the initial feature vector associated with each atom (which only inhabits angular order 0). Such embodiments use a one-hot vector which encodes if the atom is a carbon, nitrogen, or oxygen. All atoms of other element types are ignored:
Vₐ₀₀⁰=1 if atom a has element type carbon
Vₐ₁₀⁰=1 if atom a has element type oxygen
Vₐ₂₀⁰=1 if atom a has element type nitrogen

### Per-Channel Mean

After the equivariant layers, certain embodiments drop the positions of the atoms, as well as any entry of their feature vectors that do not correspond to the zeroth-order harmonic. The average can be computed, across all atoms, of each of the remaining features. This averaging produces a molecule-wide embedding that is insensitive to the original RNA's size. As only the entries corresponding to the zeroth-order harmonic are being kept, this causes further layers to be invariant to rotations, as the zeroth-order harmonic is itself invariant to rotations. This results in a new feature vector [symbol] that is indexed only by the radial channel c:

$$\\ {E}_{c} = {\sum }_{a} {V}_{ac 0}^{{}_{} 0}$$

Where W and [symbol] are a learnable weight matrix and learnable bias vector, respectively.

### Network Architecture

In total, various embodiments include 15 layers with learnable parameters (6 self-interactions, 3 equivariant convolutions, 3 pointwise non-linearities, and 3 fully connected), and 5 layers with fixed parameters (1 atomic embedding, 3 pointwise normalizations, and 1 per-channel mean) (see e.g., FIG. 8A). The first fully connected layer uses an ELU non-linearity while the other two use no non-linearities. All learnable biases were initialized to 0, and all learnable weight matrices were initialized using Xavier uniform initialization. The network was trained with the Adam optimizer to minimize the Huber loss, as applied to the difference between the predicted and true root mean square deviation (RMSD) between the atoms of the experimentally determined structure and a candidate structural model:

$$\\ R M S D = \sqrt{\frac{1}{N} {\sum }_{a} {\vert {\vec{p}}_{a} - \vec{p} {}_{a}^{{}_{} ′} \vert}^{2}}$$

Where N is the total number of atoms present, and [symbol] ₐ and [symbol] ₐ′ are the positions of atom a in the candidate model and the experimentally determined structure, respectively. RMSD calculations can be calculated by various means, including using Rosetta, excluding hydrogen atoms as well as the rare bases and sugars that make no atomic contacts in the experimentally determined structure.

Each equivariant convolution uses the real spherical harmonics of orders 0, 1, and 2, for a total of 9 angular sub-functions. The local neighborhood of an atom can be defined as the nearest 50 atoms (including the source atom itself). The overall network design, the dimension of the equivariant convolution and pointwise self-interaction layers, and the number of neurons in the dense layers are illustrated in FIG. 8A.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

What is claimed is:

1. A method for determining RNA structure, comprising:
obtaining an experimentally determined RNA structure;
training a machine learning model with the experimentally determined RNA structure;
providing an RNA sequence to the trained machine learning model; and
determining an RNA structure for the RNA sequence with the trained machine learning model.

2. The method of claim 1, wherein the machine learning model is a geometric deep learning neural network.

3. The method of claim 1, wherein the machine learning model is an equivariant neural network comprising an equivariant layer,

4. The method of claim 3, wherein the equivariant layer passes on rotational information to the next layer in the machine learning model.

5. The method of claim 3, wherein the equivariant layer passes on translational information to the next layer in the machine learning model.

6. The method of claim 3, wherein the equivariant layer comprises at least one of: a radial function and an angular function.

7. The method of claim 6, wherein the radial function encodes distances between atoms.

8. The method of claim 6, wherein the angular function considers orientations between atoms.

9. The method of claim 3, wherein the equivariant neural network further comprises at least one of a self-interaction layer, a pointwise normalization layer, a pointwise normalization layer, and a fully connected layer.

10. The method of claim 1, wherein training the machine learning model comprises sampling a training set of RNA molecules.

11. The method of claim 10, wherein the training set of RNA molecules comprises three-dimensional coordinates and chemical element type of each atom in each RNA molecule in the training set of RNA molecules.

12. The method of claim 10, wherein sampling is selected from FARFAR2 and Monte Carlo sampling.

13. The method of claim 10, wherein training the machine learning model comprises optimizing the machine learning model.

14. The method of claim 13, wherein optimizing the machine learning model comprises selecting model parameters based on a lowest root mean square deviation (RMSD) between a predicted structure and its experimentally determined structure.

15. The method of claim 10, wherein the training set comprises RNA molecules of 17-47 nucleotides.

16. The method of claim 10, wherein training the machine learning model further comprises benchmarking the machine learning model with a benchmarking set of RNA molecules.

17. The method of claim 16, wherein the benchmarking set comprises RNA molecules of 27-188 nucleotides.

18. The method of claim 1, further comprising:
obtaining a structure for a ligand; and
docking the ligand to the determined RNA structure to identify if the ligand binds to the RNA sequence.

19. The method of claim 18, further comprising providing the ligand to an individual.

20. The method of claim 1, wherein the determined RNA structure comprises both secondary and tertiary structures.
